(19) United States
(12) Patent Application Publication
(10) Pub. No.: US 2014/0180316 A1
(43) Pub. Date: Jun. 26, 2014

(54) IMAGING AND REMOVING BIOLOGICAL MATERIAL

(71) Applicant: VOLCANO CORPORATION, San Diego, CA (US)

(72) Inventor: Paul Hoseit, El Dorado Hills, CA (US)

(73) Assignee: VOLCANO CORPORATION, San Diego, CA (US)

(21) Appl. No.: 14/133,066

(22) Filed: Dec. 18, 2013

Related U.S. Application Data
(60) Provisional application No. 61/740,566, filed on Dec. 21, 2012.

(51) Int. Cl. A61B 17/3207; A61B 19/00

(52) U.S. Cl. CPC A61B 17/320783; A61B 19/5244
USPC 606/159

(57) ABSTRACT

The invention generally relates to devices and methods for imaging and removing biological material from a vessel wall. In certain embodiments, the invention provides devices that include a body configured to fit within a lumen of a vessel, the body including an opening, a biological material removal assembly configured to remove biological material that is exposed to the removal assembly via the opening, and an imaging assembly coupled to the body and positioned to image the opening.

### CROSS-REFERENCE TO RELATED APPLICATIONS

This application claims the benefit of, and priority to, U.S. Provisional Application Ser. No. 61/740,566, filed Dec. 21, 2012, the contents of which are incorporated by reference herein in its entirety.

### FIELD OF THE INVENTION

The invention generally relates to devices and methods for imaging and removing biological material from a vessel wall.

### BACKGROUND

Cardiovascular disease frequently arises from accumulation of atheromatous material on inner walls of vascular lumens, particularly arterial lumens of the coronary and other vasculature, resulting in a condition known as atherosclerosis. Atherosclerosis occurs naturally as a result of aging, but it may also be aggravated by factors such as diet, hypertension, heredity, and vascular injury. Atheromatous and other vascular deposits restrict blood flow and can cause ischemia which, in acute cases, can result in myocardial infarction. Atheromatous deposits can have widely varying properties, with some deposits being relatively soft and others being fibrous and/or calcified. In the latter case, the deposits are frequently referred to as plaque.

Atherosclerosis may be treated in a variety of ways, including drugs, bypass surgery, and a variety of catheter-based approaches that rely on intravascular debulking or removal of the atheromatous or other material occluding a blood vessel. An atherectomy catheter for use in excising material from a blood vessel lumen typically has a rotatable and/or axially translatable cutting blade or other removal mechanism or assembly, and the catheter's removal assembly is advanced into or past the occlusive material in order to cut and separate that material from the blood vessel's inner wall. One particular side-cutting atherectomy catheter is the SilverHawk atherectomy catheter (available from Covidien/EV3 and described in U.S. Pat. No. 7,927,784) which has a housing with an aperture on one side, a blade that is rotated or translated past the aperture, and a mechanism to urge the aperture against the material to be removed.

During an atherectomy procedure, an atherectomy catheter is inserted into a blood vessel and passed to the site of the obstruction. Contrast material is injected through the catheter to visualize the obstruction using an external x-ray imaging system. The catheter typically includes a radiopaque marker so that it also can be visualized by the external imaging system while the catheter is in the vessel. The catheter's removal assembly engages with the obstruction to allow removal of the atheromatous material on the inner wall of the vessel. The treatment area is visualized by the external x-ray imaging system during and subsequent to the removal to ensure that the obstruction has been removed by the catheter. If atheromatous material remains, the process is repeated until the obstruction is removed.

It is known to include an imaging sensor with an atherectomy catheter by locating the imaging sensor proximal or distal to the catheter's removal assembly (see Radvancy et al., Seminars in Interventional Radiology, 25(1), 11-19, 2008) or by integrating imaging sensor into the removal assembly (see U.S. Pat. No. 7,927,784).

### SUMMARY

The invention recognizes that a problem with known atherectomy catheters is that image sensor placement does not allow for real-time imaging of the vessel area being treated and requires moving the catheter or removal assembly back and forth to alternatively image and cut. The invention generally relates to devices and methods that allow for real-time imaging of a vessel area being treated during an atherectomy. Aspects of the invention are accomplished by providing a device with an integrated imaging assembly that is coupled to a body of the device and positioned to allow imaging of an opening in the device where a removal assembly interacts with biological material on a vessel wall. Such placement of the imaging assembly greatly improves visualization during the atherectomy procedure by allowing an operator to have real-time images of the vessel wall while the removal assembly is engaged with that portion of the vessel wall. This increases safety and allows an operator to better direct the atherectomy. Additionally, such placement of the device's imaging assembly eliminates the need for moving the device or removal assembly back and forth between imaging and cutting during an atherectomy, thereby improving efficiency of the procedure.

In certain aspects, devices of the invention include a body configured to fit within a lumen of a vessel, the body having an opening. A biological material removal assembly of the device is configured to remove biological material that is in or near the opening. An imaging assembly is coupled to the body of the device and positioned to image the opening. Devices of the present invention may be used in a variety of body lumens, including but not limited to intravascular lumens such as coronary arteries. Typically, devices of the invention are used to remove occlusive material, such as atherosclerotic plaque, from vascular lumens, but they may alternatively or also be used to remove one or more other materials.

The body of devices of the invention generally includes a proximal and a distal portion. The distal portion generally includes the opening. The opening may be located at a distal end of the body or may be located along a sidewall of the body. In certain embodiments, the opening is located on a sidewall in a distal portion of the body. The body may have any configuration that allows it to fit within a lumen of a vessel. Generally, the opening may include a slidable cover that is closed during insertion of the device into a vessel lumen, and opened once the catheter is properly positioned near an obstruction to allow the removal assembly to engage the obstruction. In certain embodiments, the device is a catheter, and the opening is located on a sidewall of the catheter. The catheter body generally includes a proximal portion and a distal portion, with the distal portion having the opening.

The removal assembly is disposed at least partially within the distal portion of the device and, in some embodiments, is radially movable to expose at least a portion of the assembly through the opening to contact the biological material in the body lumen. In catheter embodiments, the catheter may have many various sizes and configurations. In one embodiment, for example, the distal portion has an outer diameter of between about 0.1 cm and about 0.22 cm and the opening has a length of between about 0.12 cm and about 0.25 cm. The proximal portion and the distal portion of the catheter body typically define a channel having a longitudinal axis.

The removal assembly itself may take any of a number of suitable forms, but in one embodiment it includes a rotatable cutter. Optionally, such a cutter may include a beveled edge for contacting the material in the body lumen while preventing injury to the body lumen. In some embodiments, the cutter includes a tungsten carbide cutting edge for improved durability and cutting ability. In still other embodiments, the removal assembly may include a suction device, a radio frequency electrode, a laser, an ultrasound emitter and/or the like. Cutter assemblies generally employ a rotatable and/or axially translatable cutting blade that can be advanced into or past the occlusive material in order to cut and separate such material from the blood vessel lumen. In particular, side-cutting atherectomy catheters generally employ a housing having an aperture on one side, a blade which is rotated or translated by the aperture, and a balloon to urge the aperture against the material to be removed. In certain embodiments, the cutter assembly includes a rotatable auger. In embodiments including a rotatable cutter, the catheter may optionally further include a drive shaft positioned within this channel, with the drive shaft being attachable to a driver for rotating the cutter.

In devices and methods of the invention, an imaging assembly is coupled to the body and positioned to image the opening in the device. Any imaging assembly may be used with devices and methods of the invention, such as opto-acoustic sensor apparatuses, intravascular ultrasound (IVUS) or optical coherence tomography (OCT). In certain embodiments, the imaging assembly includes at least one opto-acoustic sensor. Generally, the opto-acoustic sensor will include an optical fiber having a blazed fiber Bragg grating, a light source that transmits light through the optical fiber, and a photoacoustic transducer material positioned so that it receives light diffracted by the blazed fiber Bragg grating and emits ultrasonic imaging energy. The sensor may be positioned on an internal wall of the device, opposite the opening. In certain embodiments, the at least one sensor is a plurality of sensors and the sensors are arranged in a semi-circle.

Another aspect of the invention provides methods for imaging and removing biological material from a vessel wall that involve providing a biological material removal device including a body configured to fit within a lumen of a vessel, the body having an opening, a biological material removal assembly configured to remove biological material that is exposed to the removal assembly via the opening, and an imaging assembly coupled to the body and positioned to image the opening. The method further involves inserting the device into a lumen of a vessel, and simultaneously imaging the opening while removing biological material from a vessel wall that is exposed to the removal assembly via the opening.

### BRIEF DESCRIPTION OF THE DRAWINGS

FIG. 1 shows an exemplary embodiment of a side view of a device of the invention.

FIG. 2 is a perspective view of an embodiment showing the device body and the removal assembly.

FIG. 2A is a side view of a portion of FIG. 2, where the body has a rigid distal portion with a bend, according to one embodiment of the present invention.

FIG. 3 is an exploded view of an exemplary distal portion of the device of the present invention.

FIG. 4A is an end view of the distal portion of the device of FIG. 2 in which the cutter is in a closed position in the body.

FIG. 4B is a sectional view along Line A-A of FIG. 4A.

FIGS. 4C and 4D are views of the distal portion of a device, where the distal portion has a locking shuttle mechanism.

FIG. 5A is an end view of the distal portion of the device of FIG. 2 in which the cutter is in an open position outside of the cutting window.

FIG. 5B is a sectional view along Line A-A of FIG. 5A.

FIGS. 5C and 5D are views of the distal portion of a device, where the distal portion has a locking shuttle mechanism.

FIG. 6A is an end view of the distal portion of the device FIG. 2 in which the cutter is in a packing position within a tip of the catheter.

FIG. 6B is a sectional view along Line A-A of FIG. 6A.

FIGS. 7 to 9 illustrate a monorail delivery system of the present invention.

FIG. 10A is a perspective view of a cutter of the present invention.

FIG. 10B is an end view of the cutter of FIG. 10A.

FIG. 10C is a sectional view of the cutter along Line A-A of the cutter of FIGS. 10A and 10B.

FIG. 11A is a perspective view of a in-stent restenosis cutter of the present invention.

FIG. 11B is an end view of the cutter of FIG. 11A.

FIG. 11C is a sectional view of the cutter along Line B-B of the cutter of FIGS. 11A and 11B.

FIG. 12A is a perspective view of another in-stent restenosis cutter of the present invention.

FIG. 12B is an end view of the cutter of FIG. 12A;

FIG. 12C is a sectional view of the cutter along Line C-C of the cutter of FIGS. 12A and 12B.

FIG. 12D is a side view of another embodiment of a cutter, shown partially within a catheter body.

FIG. 13 illustrates a proximal handle and cutter driver of the present invention.

FIG. 14 illustrates a cutter driver with a handle cover removed.

FIGS. 15 to 17 illustrate three positions of the lever for controlling the cutter.

FIG. 18 is a schematic diagram of a conventional optical fiber.

FIG. 19 is a cross-sectional schematic diagram illustrating generally one example of a distal portion of an imaging assembly that combines an acousto-optic Fiber Bragg Grating (FBG) sensor with an photoacoustic transducer.

FIG. 20 is a schematic diagram of a Fiber Bragg Grating based sensor

FIG. 21 is a cross-sectional schematic diagram illustrating generally one example of the operation of a blazed grating FBG photoacoustic transducer.

FIG. 22 is a schematic diagram illustrating generally one technique of generating an image by rotating the blazed FBG optical-to-acoustic and acoustic-to-optical combined transducer and displaying the resultant series of radial image lines to create a radial image.

FIG. 23 is a schematic diagram that illustrates generally one such phased array example, in which the signal to/from each array transducer is combined with the signals from the other transducers to synthesize a radial image line.

FIG. 24 is a schematic diagram that illustrates generally an example of a side view of a distal portion of a device.

FIG. 25 is a schematic diagram that illustrates generally one example of a cross-sectional side view of a distal portion of a device.

FIG. 26 is a block diagram illustrating generally one example of the imaging assembly and associated interface components.

FIG. 27 is a block diagram illustrating generally another example of the imaging assembly and associated interface components, including tissue characterization and image enhancement modules.

FIG. 28 is a simplified flow chart illustrating a method of the present invention.

FIGS. 29 and 30 illustrate a method of the present invention.

### DETAILED DESCRIPTION

The invention generally relates to devices and methods for removing biological material from a vessel wall. The devices and methods of the present invention are designed to remove tissue, such as atheroma and other occlusive material from body lumens. The body lumens generally are diseased body lumens and in particular coronary arteries. The defect in the body lumen can be a de novo lesion or an in-stent restenosis lesion for example. The devices and methods, however, are also suitable for treating stenosis of body lumens and other hyperplastic and neoplastic conditions in other body lumens, such as the ureter, the biliary duct, respiratory passages, the pancreatic duct, the lymphatic duct, and the like. Neoplastic cell growth will often occur as a result of a tumor surrounding and intruding into a body lumen. Removal of such material can thus be beneficial to maintain patency of the body lumen. The devices and methods of the present invention can collect lumenectomy samples or materials. The material obtained from the body lumen typically will be a continuous strip of tissue taken from the lumen's interior wall and ranges from about 1 mg to about 2000 mg in weight. The continuous strip or strand of tissue removed can have a length that is longer than a length of the device's cutting window or opening. While the remaining discussion is directed at removal, imaging, and passing through atheromatous or thrombotic occlusive material in a coronary artery, it will be appreciated that the systems, devices, and methods of the present invention can be used to remove and/or pass through a variety of occlusive, stenotic, or hyperplastic material in a variety of body lumens.

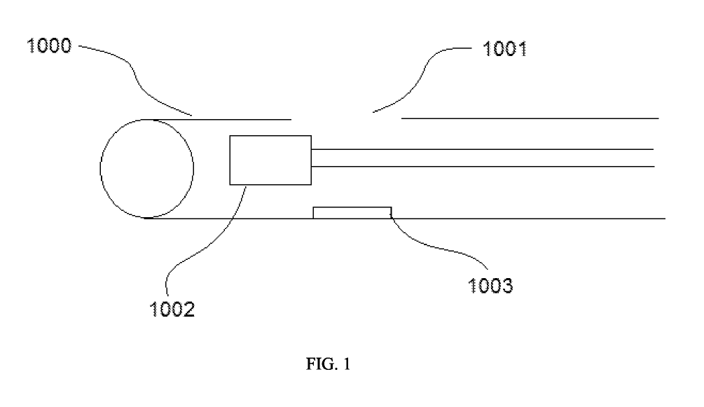

FIG. 1 shows an exemplary embodiment of a side view of a device of the invention. The device includes a body 1000 having a proximal portion and a distal portion with an opening 1001, a removal assembly 1002 that may be exposed through the opening 1001 to contact material in a body lumen, and an imaging assembly 1003 coupled to the body and positioned to image the opening 1001. The device can be used by an operator to debulk or remove biological material from a body lumen.

The body 1000 generally includes a proximal and a distal portion. The distal portion generally includes the opening 1001. The opening 1001 may be located at a distal end of the body 1000 or may be located along a sidewall of the body 1000. In certain embodiments, the opening 1001 is located on a sidewall of a distal portion of the body 1000. The body 1000 may have any configuration that allows it to fit within a lumen of a vessel. Generally, the opening 1001 may include a slidable cover (not shown) that is closed during insertion of the device into a vessel lumen, and opened once the opening 1001 is properly positioned near an obstruction.

In certain embodiments, the device is a catheter and the body is a catheter body. The catheter and catheter body are configured for intraluminal introduction to the target body lumen. The dimensions and other physical characteristics of the catheter bodies will vary significantly depending on the body lumen that is to be accessed. In the exemplary case of atherectomy catheters intended for intravascular introduction, the proximal portions of the catheter bodies will typically be very flexible and suitable for introduction over a guidewire to a target site within the vasculature. In particular, catheters can be intended for “over-the-wire” introduction when a guidewire channel extends fully through the catheter body or for “rapid exchange” introduction where the guidewire channel extends only through a distal portion of the catheter body. In other cases, it may be possible to provide a fixed or integral coil tip or guidewire tip on the distal portion of the catheter or even dispense with the guidewire entirely. For convenience of illustration, guidewires will not be shown in all embodiments, but it should be appreciated that they can be incorporated into any of these embodiments.

Catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is preferably below 8 French, more preferably below 7 French, and most preferably in the range from 2 French to 7 French. Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

The distal portion of the catheters of the present invention may have a wide variety of forms and structures. In many embodiments, a distal portion of the catheter is more rigid than a proximal portion, but in other embodiments the distal portion may be equally as flexible as the proximal portion. One aspect of the present invention provides catheters having a distal portion with a reduced rigid length. The reduced rigid length can allow the catheters to access and treat tortuous vessels and small diameter body lumens. In most embodiments a rigid distal portion or housing of the catheter body will have a diameter that generally matches the proximal portion of the catheter body, however, in other embodiments, the distal portion may be larger or smaller than the flexible portion of the catheter.

A rigid distal portion of a catheter body can be formed from materials that are rigid or which have very low flexibilities, such as metals, hard plastics, composite materials, NiTi, steel with a coating such as titanium nitride, tantalum, ME-92 (antibacterial coating material), diamonds, or the like. Most usually, the distal end of the catheter body will be formed from stainless steel or platinum/iridium. The length of the rigid distal portion may vary widely, typically being in the range from 5 mm to 35 mm, more usually from 10 mm to 25 mm, and preferably between 6 mm and 8 mm. In contrast, conventional catheters typically have rigid lengths of approximately 16 mm.

The side opening windows of the present invention will typically have a length of approximately 2 mm. In other embodiments, however, the side opening cutting window can be larger or smaller, but should be large enough to allow the cutter to protrude a predetermined distance that is sufficient to remove material from the body lumen.

The catheter may include a flexible atraumatic distal tip coupled to the rigid distal portion of the catheter. For example, an integrated distal tip can increase the safety of the catheter by eliminating the joint between the distal tip and the catheter body. The integral tip can provide a smoother inner diameter for ease of tissue movement into a collection chamber in the tip. During manufacturing, the transition from the housing to the flexible distal tip can be finished with a polymer laminate over the material housing. No weld, crimp, or screw joint is usually required.

The atraumatic distal tip permits advancing the catheter distally through the blood vessel or other body lumen while reducing any damage caused to the body lumen by the catheter. Typically, the distal tip will have a guidewire channel to permit the catheter to be guided to the target lesion over a guidewire. In some exemplary configurations, the atraumatic distal tip includes a coil. In some configurations the distal tip has a rounded, blunt distal end. The catheter body can be tubular and have a forward-facing circular aperture which communicates with the atraumatic tip. A collection chamber can be housed within the distal tip to store material removed from the body lumen. The combination of the rigid distal end and the flexible distal tip is approximately 30 mm.

Devices of the invention include a removal assembly. Any removal assembly known in the art may be used with devices of the invention. Exemplary removal assemblies include a suction device, a radio frequency electrode, a laser, an ultrasound emitter and/or the like. In particular embodiments, the removal assembly includes a cutter apparatus. Optionally, such a cutter may include a beveled edge for contacting the material in the body lumen while preventing injury to the body lumen. In some embodiments, the cutter includes a tungsten carbide cutting edge for improved durability and cutting ability. Cutter assemblies generally employ a rotatable and/or axially translatable cutting blade that can be advanced into or past the occlusive material in order to cut and separate such material from the blood vessel lumen. In particular, side-cutting atherectomy catheters generally employ a housing having an aperture on one side, a blade which is rotated or translated by the aperture, and a balloon to urge the aperture against the material to be removed. In certain embodiments, the cutter assembly includes a rotatable auger. In embodiments including a rotatable cutter, the catheter may optionally further include a drive shaft positioned within this channel, with the drive shaft being attachable to a driver for rotating the cutter.

A rotatable cutter or other removal assembly may be disposed in the distal portion of the catheter to sever material that is adjacent to or received within the cutting opening. In an exemplary embodiment, the cutter is movably disposed in the distal portion of the catheter body and movable across a side opening window. A straight or serrated cutting blade or other element can be formed integrally along a distal or proximal edge of the cutting window to assist in severing material from the body lumen. In one particular embodiment, the cutter has a diameter of approximately 1.14 mm. It should be appreciated however, that the diameter of the cutter will depend primarily on the diameter of the distal portion of the catheter body.

In exemplary embodiments, activation of an input device can deflect a distal portion of the catheter relative to the proximal portion of the catheter. Angular deflection of the distal portion may serve one or more purposes in various embodiments. Generally, for example, deflection of the distal portion increases the effective “diameter” of the catheter and causes the removal assembly to be urged against material in a lumen, such as, but not limited to, atherosclerotic plaque. In other embodiments, deflection of the distal portion may act to expose a removal assembly through a window for contacting material in a lumen. In some embodiments, for example, activation of the input device moves the debulking assembly over a ramp or cam so that a portion of the rigid distal portion and flexible tip are caused to drop out of the path of the removal assembly so as to expose the removal assembly through the opening. In some embodiments, deflection may both urge a portion of the catheter into material in a lumen and expose a tissue removal assembly.

It should be understood that movement of a tissue removal assembly may cause deflection of a portion of the catheter or that deflection of the catheter may cause movement or exposure of a tissue removal assembly, in various embodiments. In other embodiments, deflection of a portion of the catheter and movement of the tissue removal assembly may be causally unconnected events. Any suitable combination of deflecting, exposing of a debulking assembly and the like is contemplated. In carrying out deflection, exposure and/or the like, a single input device may be used, so that a user may, for example, deflect a portion of a catheter and expose a tissue removal assembly using a single input device operable by one hand. In other embodiments, rotation of a tissue removal assembly may also be activated by the same, single input device. In other embodiments, multiple input devices may be used.

Some embodiments further help to urge the removal assembly into contact with target tissue by including a proximal portion of the catheter body having a rigid, shaped or deformable portion. For example, some embodiments include a proximal portion with a bend that urges the tissue assembly toward a side of the lumen. In other embodiments, one side of the proximal portion is less rigid than the other side. Thus, when tension is placed on the catheter in a proximal direction (as when pulling the removal assembly proximally for use), one side of the proximal portion collapses more than the other, causing the catheter body to bend and the removal assembly to move toward a side of the lumen.

In exemplary embodiments, the removal assembly includes a rotatable cutter that is movable outside the opening. By moving the cutter outside of the cutting opening beyond an outer diameter of the distal portion of the catheter, the cutter is able to contact and sever material that does not invaginate into the cutting window. In a specific configuration, the rotating cutter can be moved over the cam within the rigid, or distal, portion of the catheter body so that the cutting edge is moved out of the opening. Moving the rotating cutter outside of the cutting opening and advancing the entire catheter body distally, a large amount of occlusive material can be removed. Consequently, the amount of material that can be removed is not limited by the size of the cutting opening.

The material or tissue excised from the body lumen will vary in length and will depend on the catheter configuration, the type of material removed, the body lumen, and the like. However, in certain embodiments, the material will be in the form of continuous strands that has a substantially consistent depth and width of tissue cuts. The material is typically longer than the length of the cutting opening (but it may be shorter), and typically has a length of about 2.0 mm or longer, and sometimes between about 0.5 cm up to about 10 cm or longer in length. Typically the length of a continuous strand is at least 2 cm, at least 5 cm, at least 7 cm, at least 10 cm, or at least 15 cm. The length of a strand is the dimension which is axial to the lumen. Advantageously, the planing action of the catheter provides a material tissue structure that reflects the actual in vivo tissue structure, and provides information about larger portions of the disease state of the body lumen. One or more strands may be obtained from a single vascular lumen or single vascular obstruction. Because of the design and configuration of the device, the strands typically have a depth of at least 0.1 mm, at least 0.25 mm, at least 0.33 mm, or at least 0.5 mm. Depth of a strand is the dimension which is radial to the axis of a lumen. The cutting and planing action of the device of the invention achieves large volumes which are excellent for analysis, for multiple analyses, for storage as archival samples, and for assembly into libraries of samples representative of certain disease states.

The mass/length ratio of continuous strands is typically at least 0.45 mg/mm, at least 0.50 mg/mm, at least 0.55 mg/mm, at least 0.60 mg/mm, at least 0.65 mg/mm, or at least 0.70 mg/mm. The samples can be preserved according to any method known in the art. Samples may be frozen, for example, in liquid nitrogen, they may be preserved in paraffin, dried, freeze dried, etc. Samples may be treated to achieve a purified or semi-purified component of the sample. Samples may be treated, for example to extract DNA or protein. Samples may be treated to extract mRNA and to preserve it or “convert” it to cDNA. Desirably, samples are stored in a systematic way so that patient information remains associated with the samples and patient outcome can be associated with the sample concurrently or at a later time.

The material removed from the collection chamber, or a portion thereof, can be placed in a preserving agent, a tissue fixative, and or a preparation agent suitable for a desired test prior to testing the material. The material removed from the patient by this method is typically at least one or more continuous strip(s) of material that maintains the structure of the material in vivo. The quantity of material removed by the method can be from about 1 mg to about 2000 mg. Typically the amount of material is about 1 mg to about 100 mg, about 100 mg to about 200 mg, about 200 mg to about 300 mg, 300 mg to about 400 mg, 400 mg to about 500 mg, 500 mg to about 600 mg, about 600 mg to about 700 mg, 700 mg to about 800 mg, or about 800 mg to about 2000 mg. In a typical procedure about 400 mg to about 600 mg of material is removed and available for testing and/or storage. A preferred embodiment of the present invention provides for the collection of one or more continuous strips of material from the inner surface of the lumen that is longer than a largest dimension of the cutting window. In a particular example, the material can comprise plaque tissue.

As will be described in detail below, in some situations it is preferable to provide a serrated cutting edge, while in other situations it may be preferable to provide a smooth cutting edge. Optionally, the cutting edge of either or both the blades may be hardened, e.g., by application of a coating. A preferred coating material is a chromium based material, available from ME-92, Inc., which may be applied according to manufacturer's instructions. In some embodiments, the cutter includes a tungsten carbide cutting edge. Other rotatable and axially movable cutting blades are described in U.S. Pat. Nos. 7,927,784; 5,674,232; 5,242,460; 5,312,425; 5,431,673; and 4,771,774, the full disclosures of which are incorporated herein by reference. In some embodiments, a rotatable cutter includes a beveled edge for removal of material from a body lumen while preventing injury to the lumen. In still other embodiments, a tissue removal assembly may include alternative or additional features for removing biological material from a lumen. For example, the removal assembly may include, but is not limited to, a radio frequency device, an abrasion device, a laser cutter and/or the like.

The devices of the present invention may include a monorail delivery system to assist in positioning the cutter at the target site. For example, the tip of the catheter can include lumen(s) that are sized to receive a conventional guidewire (typically 0.014″ diameter) or any other suitable guidewire (e.g., having diameters between 0.018″ and 0.032″) and the flexible proximal portion of the catheter body can include a short lumen (e.g., about 12 centimeters in length). Such a configuration moves the guidewire out of the rigid portion so as to not interfere with the removal assembly.

In other embodiments, however, the guidewire lumen may be disposed within or outside the flexible proximal portion of the catheter body and run a longer or shorter length, and in fact may run the entire length of the flexible portion of the catheter body. The guidewire can be disposed within lumen on the flexible portion of the catheter body and exit the lumen at a point proximal to the rigid portion of the catheter. The guidewire can then enter a proximal opening in the tip lumen and exit a distal opening in the tip lumen. In some embodiments, the catheter has a distal guidewire lumen on its flexible distal tip and a proximal guidewire lumen on its flexible body. For example, in some embodiments the distal lumen may have a length of between about 2.0 cm and about 3.0 cm and the proximal lumen may have a length of between about 10 cm and about 14 cm. In yet further embodiments, a distal tip guidewire lumen may be configured to telescope within a proximal guidewire lumen, or vice versa. A telescoping guidewire lumen may enhance performance of the catheter by preventing a guidewire from being exposed within a body lumen.

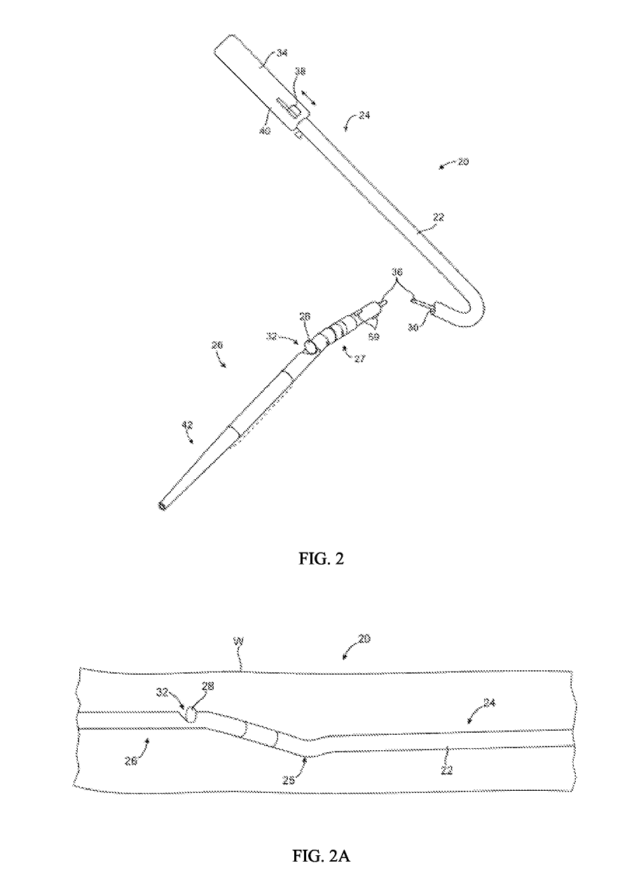

An exemplary embodiment of a device including a removal assembly is shown in FIG. 2. For illustration purposes, this figure does not show the imaging assembly. In FIG. 2, the device is illustrated as a catheter. FIG. 2 shows catheter 20 that includes a catheter body 22 having a proximal portion 24 and a distal portion 26. Proximal portion 24 can be coupled to distal portion 26 with a connection assembly 27 to allow pivoting or deflection of distal portion 26 relative to proximal portion 24. A proximal end of the catheter body 22 can have a handle 40 for manipulation by a user, a luer for connection to an aspiration or fluid delivery channel, or the like.

A removal assembly, such as a cutter 28, abrasive member, or the like, is disposed within a lumen 30 of the catheter body 22. The cutter 28 is typically rotatable within the distal portion 26 about an axis that is parallel to the longitudinal axis of the distal portion 26 of catheter 20 and axially movable along the longitudinal axis. The cutter 28 can access target tissue through a side opening window 32 that is typically large enough to allow the cutter 28 to protrude through and move out of the window 32 a predetermined distance. The cutter is coupled to a cutter driver 34 through a coiled drive shaft 36. Actuation of a movable actuator or other input device 38 can activate the drive shaft 36 and cutter, move cutter 28 longitudinally over a cam so as to deflect the distal portion and move the cutter 28 out of cutting window 32. Camming of the cutter 28 can cause the distal portion 26 to pivot or deflect relative to the proximal portion 24 so as to deflect and urge the cutter into the tissue in the body lumen.

In some embodiments, the distal portion 26 of the catheter may be moved to an angled or offset configuration from the longitudinal axis of the proximal portion 24 of the catheter and the cutter 28. In some embodiments, the cutter 28 can also be deflected off of the axis of the proximal and/or distal portion of the catheter. Moving the distal portion 26 to an angled/offset position may cause a portion of the catheter to urge against a target tissue, may expose the cutter 28 through the opening 32 or both, in various embodiments.

In catheters 20, proximal portion 24 is typically relatively flexible and distal portion 26 is typically relatively rigid. Additionally, many embodiments include a flexible distal tip 42. The flexible proximal portion 24 of the catheter is typically a torque shaft and the distal portion 26 is typically a rigid tubing. The torque shaft 24 facilitates transportation of the catheter body 22 and cutter 28 to the target tissue, e.g., diseased site. The proximal end of the torque shaft 24 is coupled to a proximal handle 40 and the distal end of the torque shaft is attached to the distal, rigid portion 26 of the catheter through the connection assembly 27. The drive shaft 36 is movably positioned within the torque shaft 24 so as to rotate and axially move within the torque shaft 24. The drive shaft 36 and torque shaft 24 are sized to allow relative movement of each shaft without interfering with the movement of the other shaft. The catheter body will have the pushability and torqueability such that torquing and pushing of the proximal end will translate motion to the distal portion 26 of the catheter body 22.

Referring now to FIG. 2A, a catheter 20 as in FIG. 2 may have a flexible proximal portion 24 that additionally includes urging means 25. As shown in FIG. 2A, urging means 25 may include a rigid bent or curved shape towards the distal end of proximal portion 24, which may help urge the cutter 28 or other removal apparatus toward a wall of a body lumen to enhance treatment. Such a rigid bend increases the working range of the catheter by allowing the cutter to be urged into a lumen wall across a wider diameter lumen.

In other embodiments, urging means 25 may take many other suitable forms. For example, a similar result to the rigid bend may be achieved by including a rigid distal portion that is not permanently bent but that is more rigid on one side than on the opposite side of catheter body 22. Thus, when proximal tension is applied to the proximal portion 24, as when proximal force is applied to the removal apparatus to expose the cutter 28 through the window 32, the urging means 25 (i.e., the rigid distal portion of proximal portion 24) will cause the catheter body 22 to bend toward the less rigid side. The less rigid side will typically be the same side as the opening 32, so that the opening 32 and/or the cutter 28 will be urged against a wall of a body lumen by the bend. In still other embodiments, a shaped element may be introduced into catheter body to act as urging means 25. Any suitable urging means is contemplated.

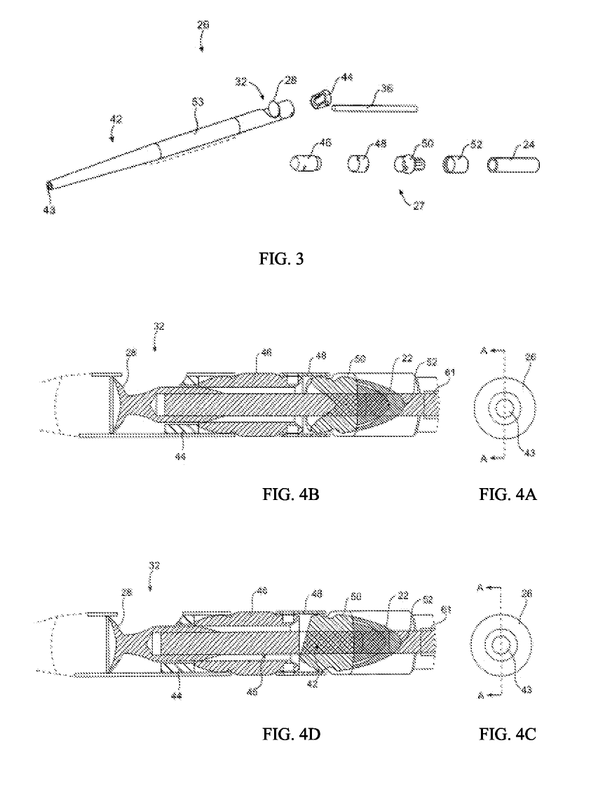

FIG. 3 illustrates an exploded view of a distal end of the catheter. In such embodiments, the catheter 20 includes a connection assembly 27, a rigid housing 26, a distal tip 42 that at least partially defines a collection chamber 53 for storing the severed atheromatous material, and a lumen that can receive the guidewire. The distal tip 42 can have a distal opening 43 that is sized to allow an imaging guidewire or conventional guidewire (not shown) to be advanced distally through the tip. In some embodiments, the distal tip 42 may also include a distal guidewire lumen (not shown) for allowing passage of a guidewire. For example, some embodiments may include a distal guidewire lumen having a length of between about 1.0 cm and about 5.0 cm, and preferably between about 2.0 cm and about 3.0 cm. Such a distal guidewire lumen may be used alone or in conjunction with a proximal guidewire lumen located on another, more proximal, portion of the catheter 20.

In embodiments including a distal guidewire lumen and a proximal guidewire lumen, the distal lumen may be configured to partially telescope within a portion of the proximal guidewire lumen, or vice versa. Such telescoping lumens may be used in embodiments where the distal portion 26 of catheter body 22 is movable relative to the proximal portion 24. A telescoping lumen may enhance performance of the catheter 20 by allowing a guidewire to be maintained largely within a lumen and to not be exposed within the body lumen being treated. Telescoping lumens may have any suitable diameters and configurations to allow for sliding or otherwise fitting of one lumen within another.

As mentioned above, various embodiments of the invention may allow for deflection of a portion of a catheter, exposure of a tissue removal assembly through an opening, or both. In some embodiments, movement of a biological material removal assembly causes deflection of a portion of the catheter. In other embodiments, deflection of the catheter may cause a tissue removal assembly to be exposed through an opening on the catheter. In still other embodiments, there may be no causal relationship between deflection of the catheter and exposure of the removal assembly, i.e., they may be separately caused.

As an example, a ramp or cam 44 may at least partially fit within the distal portion 26. As will be described in detail below, in some embodiments proximal movement of the cutter 28 over the ramp 44, causes the deflection of the distal housing 26 and guides cutter 28 out of cutting opening 32. In other embodiments, a ramp may be used to deflect the distal portion without extending the cutter out of the opening. Attached to the ramp 44 is a housing adaptor 46 that can connect one or more articulation member 48 to the distal tip to create an axis of rotation of the distal portion 26. The housing adaptor 46 and articulation member 48 allow the distal end of the catheter to pivot and bias against the body lumen. In the illustrated embodiment there are only one housing adaptor 46 and one articulation member 48, but it should be appreciated that the catheters of the present invention can include, two, three, or more joints (e.g., axis of rotation), if desired. Moreover, the axes of rotation can be parallel or non-parallel with each other.

The catheter can also include a shaft adaptor 50 and collar 52 to couple articulation member 48 to the torque shaft 22. Shaft adaptor 50 can connect the housing to the torque shaft and collar 52 can be placed over a proximal end of the shaft adaptor and crimped for a secure attachment. It should be appreciated by one of ordinary skill in the art that that while one exemplary catheter of the present invention has the above components that other catheters of the present invention may not include more or fewer of the components described above. For example, some components can be made integral with other components and some components may be left out entirely. Thus, instead of having a separate ramp 44, the ramp may be integrated with the distal tip to direct the cutter out of the cutting window.

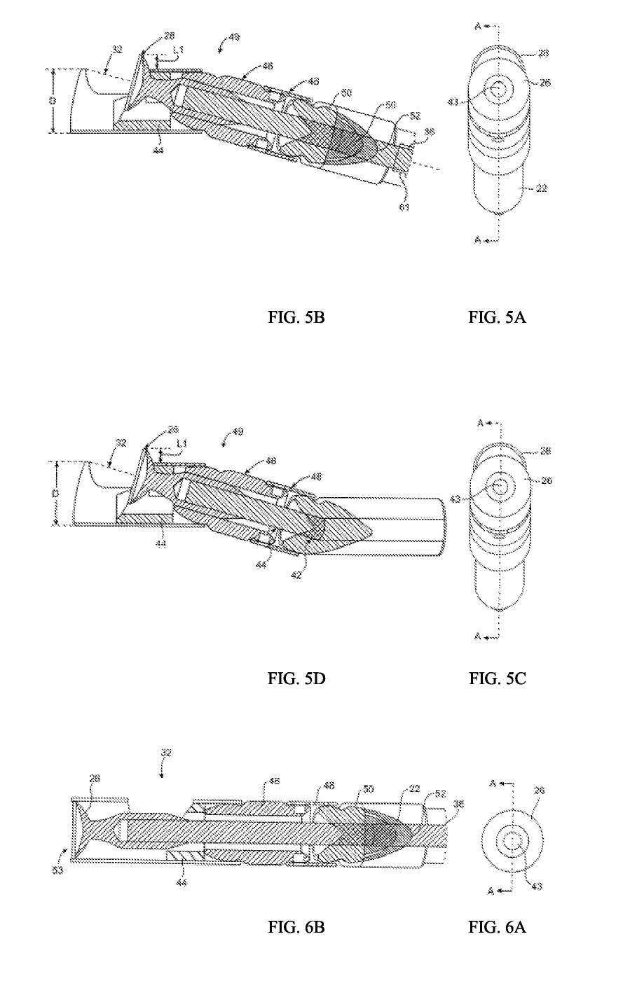

As shown in FIGS. 4-6, the cutters 28 of the present invention will generally be movable between two or more positions. During advancement through the body lumen, the cutter will generally be in a neutral position (FIGS. 4A and 4B) in which the cutter 28 is distal of cutting window 32. Once the catheter 20 has reached the target site, the cutter 28 can be moved to an open position (FIGS. 5A and 5B) in which the cutter 28 is moved to a proximal end of the cutting opening 32 and will extend out of the cutting opening 32 a distance L1 beyond an outer diameter D of the rigid portion 26. In most embodiments, in the open position, the cutter will have deflected the distal portion and the cutter's axis of rotation will generally be in line with connection assembly 27 but angled or offset from longitudinal axis of the distal portion of the catheter body.

Optionally, in some embodiments, cutter 28 can be moved to a packing position, in which the cutter is moved distally, past the neutral position, so as to pack the severed tissue into a distal collection chamber 53 (FIGS. 6A and 6B). It should be appreciated however, that while the exemplary embodiment moves the cutter to the above described positions, in other embodiments of the present invention the cutter can be positioned in other relative positions. For example, instead of having the neutral position distal of the cutting window, the neutral position may be proximal of the window, and the open position may be along the distal end of the cutting window, or the like.

Referring again to FIGS. 5A and 5B, the interaction of the components of the rigid distal portions 26 in one exemplary embodiment of the present invention will be further described. As shown in FIG. 5B, the cutting opening 32 is typically a cutout opening in the distal portion 26. While the size of the cutting opening 32 can vary, the cutting opening should be long enough to collect tissue and circumferentially wide enough to allow the cutter to move out of the cutting window during cutting, but sized and shaped to not expel emboli into the vasculature. Cams or ramp 44 (shown most clearly in FIG. 5B) can be disposed in the distal portion of the catheter body to guide or otherwise pivot the cutter 28 out of the cutting window 32 as the cutter 28 is pulled proximally through tensioning of drive shaft 36.

A joint is located proximal to the cutting opening 32 to provide a pivot point for camming of the distal portion 26 relative to the proximal portion 24. The bending at a flexible joint 49 is caused by the interaction of cams or ramps 44 with cutter 28 and the tensile force provided through drive shaft 36. In the exemplary configuration, the joint includes a housing adaptor 46 that is pivotally coupled to the distal rigid portion 26. As shown in FIGS. 5A and 5B, the resulting pivoting of the rigid distal portion 26 relative to the proximal portion causes a camming effect that urges the distal housing against the body lumen wall without the use of urging means (e.g., a balloon) that is positioned opposite of the cutting window. Thus, the overall cross sectional size of the catheter bodies can be reduced to allow the catheter to access lesions in smaller body lumens. In exemplary embodiments, the distal housing can deflect off of the axis of the proximal portion of the catheter typically between 0° and 30°, usually between 5° and 20°, and most preferably between 5° and 10°. The angle of deflection relates directly to the urge. Urge, however, does not necessarily relate to force but more to the overall profile of the catheter. For example, the greater the angle of deflection, the larger the profile and the bigger the lumen that can be treated. The ranges were chosen to allow treatment of vessels ranging from less than 2 mm to greater than 3 mm within the limits of mechanical design of the components. It should be appreciated however, that the angles of deflection will vary depending on the size of the body lumen being treated, the size of the catheter, and the like.

In some embodiments, the deflection of the distal portion 26 of the catheter urges the cutter into position such that distal advancement of the entire catheter body can move the rotating cutter through the occlusive material. Because the cutter is moved a distance L1 beyond the outer diameter of the distal portion of the catheter and outside of the cutting opening, the user does not have to invaginate the tissue into the cutting opening. In some embodiments, for example, the cutter can be moved between about 0.025 mm and about 1.016 mm, and preferably between about 0.025 mm and about 0.64 mm, beyond the outer dimension of the distal housing. It should be appreciated that the cutter excursion directly relates to the depth of cut. The higher the cutter moves out of the cutting opening the deeper the cut. The ranges are chosen around efficacy without risk of perforation of the body lumen.

Some embodiments of the catheter include a shuttle mechanism or other similar mechanism for temporarily locking the catheter in a cutting position. FIGS. 4C and 4D illustrate such an embodiment in the neutral, non-cutting position. Such embodiments generally include a shuttle member 45 and a shuttle stop member 42. The shuttle stop member 42 is typically disposed at an angle, relative to a longitudinal axis through the catheter. FIGS. 5C and 5D show the same embodiment in the cutting position. When the cutter 28 is moved into the cutting position in such embodiments, the shuttle member 45 falls into the shuttle stop member 42 and thus locks the removal apparatus in a cutting position. To unlock the removal apparatus, the cutter 28 may be advanced forward, distally, to release the shuttle member 45 from the shuttle stop member 42.

Some embodiments including a shuttle mechanism will also include two joints in catheter body 22. Thus, catheter body 22 will include a proximal portion 26, a distal portion 24 and a middle portion. When shuttle mechanism is activated to expose cutter 28 through window 32, the middle portion may orient itself at an angle, relative to the proximal and distal portions, thus allowing the removal assembly to be urged towards a side of a lumen. Such a two-jointed configuration may provide enhanced performance of the catheter 20 by providing enhanced contact of the cutter 28 with biological material to be removed from a body lumen.

Pushing the entire catheter across a lesion removes all or a portion of the lesion from the body lumen. Severed tissue from the lesion is collected by directing it into a collection chamber 53 in the tip via the cutter 28. Once the catheter and cutter 28 have moved through the lesion, the cutter 28 can be advanced distally to a “part off position” in which the cutter is moved back into the cutting opening 32 (FIG. 4B). The tissue is collected as the severed pieces of tissue are directed into a collection chamber 53 via the distal movement of cutter 28 and catheter. The collection chamber 53 of the tip and distal portion 26 acts as a receptacle for the severed material, to prevent the severed occlusive material from entering the body lumen and possibly causing downstream occlusions. The cutter 28 can interact with the distal edge of the cutting window to part off the tissue and thereafter pack the severed tissue into collection chamber 53 (FIG. 4B). In exemplary embodiments, the driver motor can be programmed to stop the rotation of the cutter at the part off position so that the cutter 28 can move to a third position (FIG. 6B) and pack the material in the collection chamber in the tip without rotation. Typically, the collection chamber 53 will be large enough to allow multiple cuts to be collected before the device has to be removed from the body lumen. When the collection chamber is full, or at the user's discretion, the device can be removed, emptied and reinserted over the guidewire via a monorail system, as will be described below.

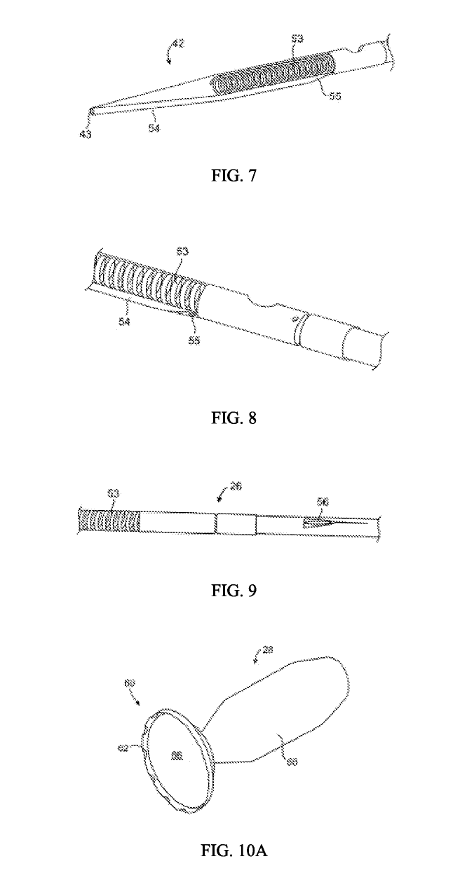

In some embodiments, the collection chamber 53 may connect to the rigid housing by means of interlocking components, which interlock with complementary components on the rigid housing. Such components may resemble a screw-in configuration, for example. Interlocking components will provide a stable connection between the collection chamber 53 and the rigid housing while not increasing the outer diameter of either the chamber 53 or the housing. Generally, collection chamber 53 may be given any suitable configuration, shape or size. For example, collection chamber 53 in FIGS. 7-9 has a helical configuration. Alternatively, collection chamber 53 may include a series of circular members, straight linear members, one solid cylindrical or cone-shaped member or the like.

FIGS. 7 through 9 illustrate one exemplary monorail delivery system to assist in positioning the cutter 28 at the target site. For example, tip 42 of the catheter can include a lumen 54 having a distal opening 43 and a proximal opening 55 that is sized to receive a guidewire, having a diameter of about 0.014 in., about 0.018 in., about 0.032 in. or any other suitable diameter.

As shown in FIG. 9, the flexible proximal portion of the catheter body may also include a short lumen 56 (e.g., about 12 centimeters in length). In some embodiments, however, the guidewire lumen 56 may be disposed within or outside the flexible proximal portion of the catheter body and run a longer or shorter length, and in fact may run the entire length of the flexible portion 24 of the catheter body. In use, the guidewire can be disposed within lumen 56 on the flexible portion of the catheter body and exit the lumen at a point proximal to the rigid portion 26 of the catheter. The guidewire can then re-enter a proximal opening 55 in the tip lumen 54 and exit through distal opening 43 in the tip lumen. By moving the guidewire outside of the rigid portion 26 of the catheter body, the guidewire will be prevented from tangling with the cutter 28. Typically, tip lumen 54 will be disposed along a bottom surface of the tip and the lumen 56 will be disposed along a side of the proximal portion 22 of the catheter body so that the guidewire will be in a helical configuration. In various embodiments, the tip lumen 54 and the proximal lumen 56 can have any suitable combination of lengths. For example, in one embodiment the tip lumen 54 may have a length between about 1 cm and about 5 cm, more preferably between about 2 cm and about 3 cm, and the proximal lumen may have a length of between about 8 cm and about 20 cm, more preferably between about 10 cm and about 14 cm.

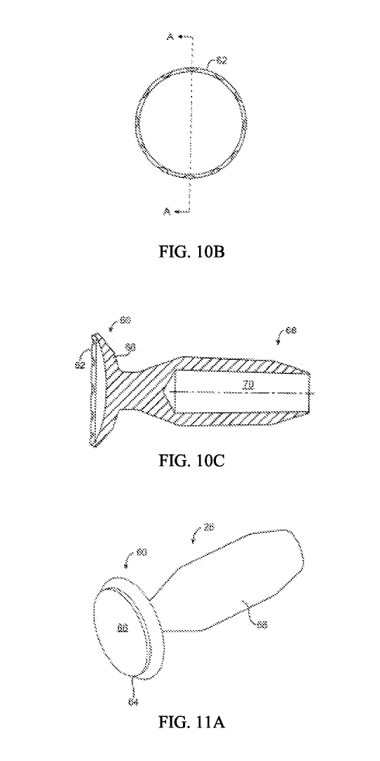

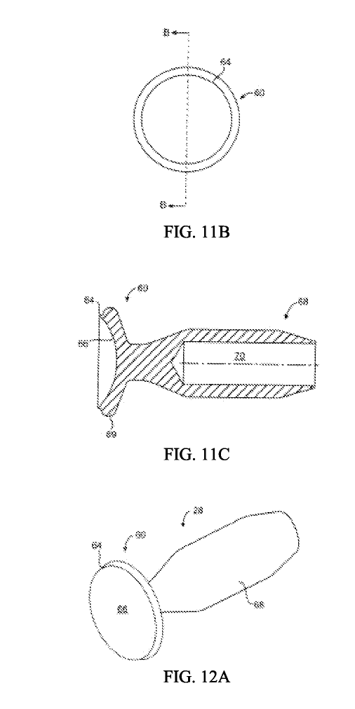

FIGS. 10A through 12D show some exemplary embodiments of the cutter 28 of the present invention. The distal portion 60 of the rotatable cutter 28 can include a serrated knife edge 62 or a smooth knife edge 64 and a curved or scooped distal surface 66. The distal portion 60 may have any suitable diameter or height. In some embodiments, for example, the diameter across the distal portion 60 may be between about 0.1 cm and about 0.2 cm. A proximal portion 68 of the cutter 28 can include a channel 70 that can be coupled to the drive shaft 36 that rotates the cutter. As shown in FIGS. 11A-11C, some embodiments of the cutters can include a bulge or bump 69 that is provided to interact with a stent so as to reduce the interaction of the cutting edge with the stent. In any of the foregoing embodiments, it may be advantageous to construct a serrated knife edge 62, a smooth knife edge 64, or a scooped distal surface 66 out of tungsten carbide.

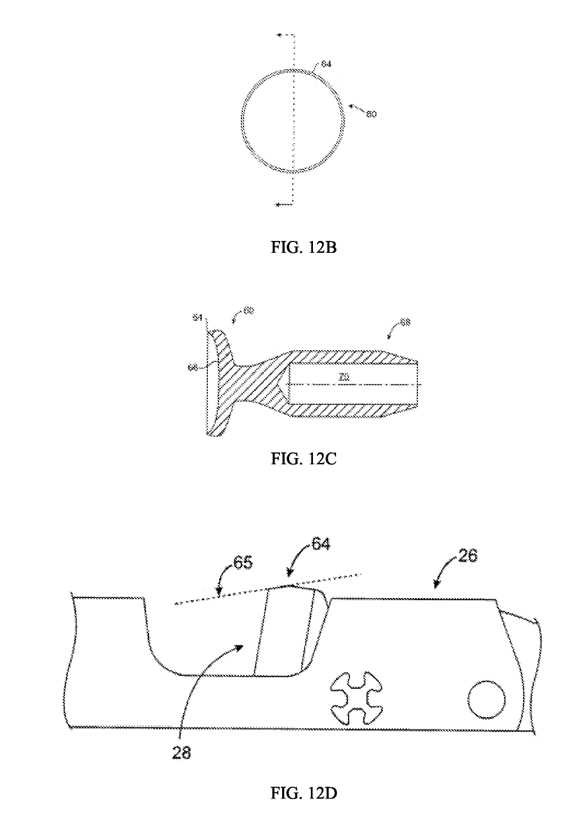

Another embodiment of a cutter 28 suitable for use in the present invention is shown in side view within a catheter body distal portion 26 in FIG. 12D. In this embodiment, the cutter 28 has a beveled edge 64, made of tungsten carbide, stainless steel, titanium or any other suitable material. The beveled edge 64 is angled inward, toward the axis of rotation (or center) of the cutter 28, creating a “negative angle of attack” 65 for the cutter 28. Such a negative angle of attack may be advantageous in many settings, when one or more layers of material are desired to be removed from a body lumen without damaging underlying layers of tissue. Occlusive material to be removed from a vessel typically has low compliance and the media of the vessel (ideally to be preserved) has higher compliance. A cutter 28 having a negative angle of attack may be employed to efficiently cut through material of low compliance, while not cutting through media of high compliance, by allowing the high-compliance to stretch over the beveled surface of cutter 28.

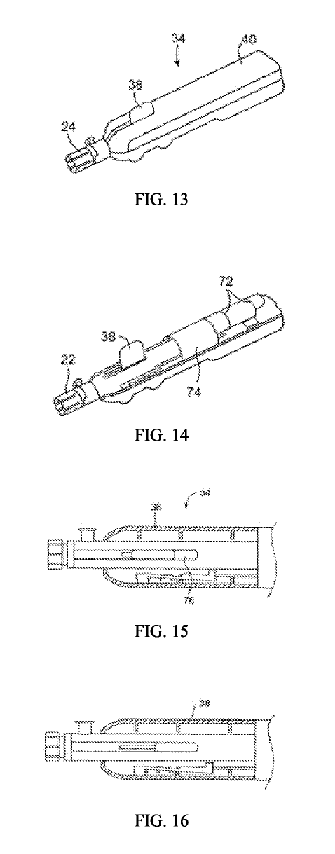

FIGS. 13 through 17 illustrate an exemplary cutter driver 34 of the present invention. As shown in FIGS. 13 and 14, cutter driver 34 can act as the handle for the user to manipulate the catheters 20 of the present invention as well as a power source. Typically, the cutter drivers 34 of the present invention include a single input device, such as a lever 38 that controls the major operations of the catheter (e.g., axial movement to cause urging, rotation to cause cutting, and axial movement for packing). As shown in FIGS. 14 and 15, cutter driver 34 includes a power source 72 (e.g., batteries), a motor 74, a microswitch 76 for activating motor 74, and a connection assembly (not shown) for connecting the drive shaft 36 to the driver motor 74. In some embodiments, the drive motor can rotate drive shaft 36 between 1,000 rpm and 10,000 rpm or more, if desired.

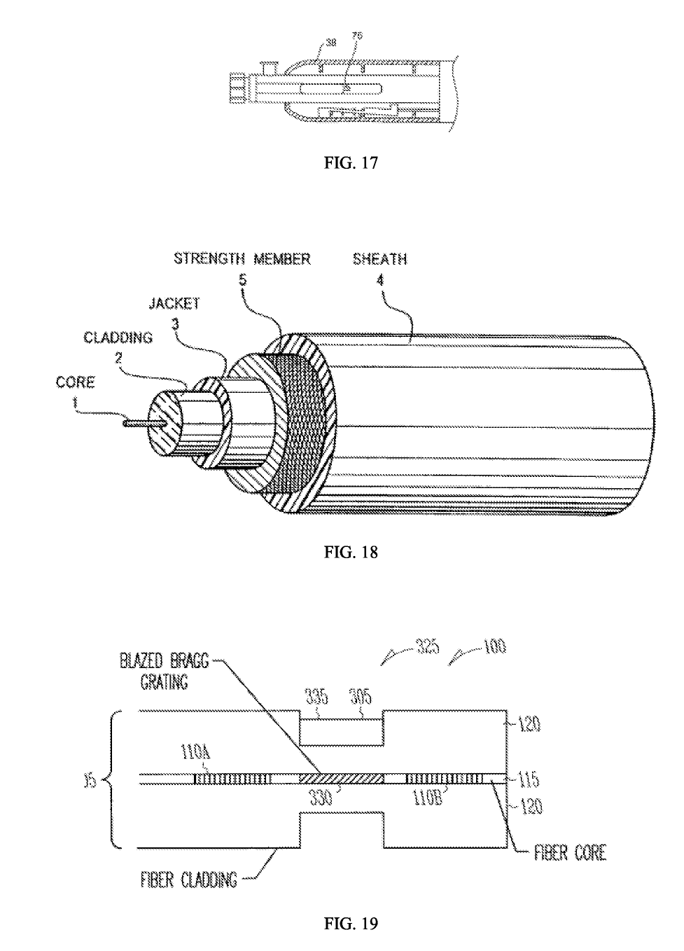

FIGS. 15 through 17 illustrate one exemplary method of operating cutter driver 34. In use, the catheter will be delivered to the target site with cutter driver unattached and the cutter in the neutral position (FIG. 4B). The cutter driver can be attached with the urge lever 38 in a neutral position (FIG. 15), which indicates that the cutter is closed, but not in a packing position. The user can then move the catheter (and cutter driver unit, if desired) to position the distal portion 26 of the catheter adjacent the target tissue. As shown in FIG. 16, to activate the rotation of the cutter, the urge lever 38 can be moved proximally from the neutral position to move the cutter proximally and out of cutting window 32 (FIG. 5B) and simultaneously depressing microswitch 76 to activate motor 74. At the end of the cutting procedure, as shown in FIG. 17, the user can push urge lever 38 completely forward to a distal position to push the cutter into a packing position (FIG. 6B). After the urge lever passes the middle of the travel, the microswitch 76 can be released so as to deactivate the cutter before reaching the packing position such that packing can occur without the cutter rotating. It should be appreciated, while the figures illustrate the use of an urge lever or thumb switch as an input device, the present invention can use other type of input devices, such as labeled buttons (e.g., close opening, remove tissue, and pack), or the like.

Advantageously, cutter driver 34 provides an automatic on/off control of the cutter 28 that is keyed to the position of the cutter. Such a configuration frees the user from the complicated task of remembering the sequence of operations to activate and deactivate the rotation and axial movement of the cutter.

While the cutter driver 34 is illustrated as a disposable battery powered unit, it should be appreciated that in other embodiments, the cutter driver can use other power sources to control the cutter driver. It should further be appreciated that other cutter drivers can be used with the present invention. While not preferred, it is possible to have separate controls to control the axial movement of the cutter and the rotation of the cutter.

Devices of the invention also include an imaging assembly coupled to the body and positioned to imaging the opening in the device. In this manner, the imaging assembly can image the interaction of the removal assembly with tissue that is exposed to the imaging assembly via the opening. Such placement of the imaging assembly improves visualization during the atherectomy procedure, allowing an operator to having real-time visualization of a vessel wall while the removal assembly is engaged, thereby increasing safety and allowing an operator to better direct the atherectomy. Additionally, such placement eliminates the need for moving a device back and forth between imaging and cutting during an atherectomy, thereby improving efficiency of the procedure. Even further, such a combination allows for real-time monitoring of biological material entering the collection chamber. By facilitating the assessment of collection chamber filling, these embodiments will reduce the need for manually withdrawing the catheter to examine the collection chamber.

Any imaging assembly may be used with devices and methods of the invention, such as optical-acoustic imaging apparatus, intravascular ultrasound (IVUS) or optical coherence tomography (OCT). In certain embodiments, the imaging assembly is an optical-acoustic imaging apparatus. Exemplary optical-acoustic imaging sensors are shown for example in, U.S. Pat. No. 7,245,789; U.S. Pat. Nos. 7,447,388; 7,660,492; U.S. Pat. No. 8,059,923; US 2012/0108943; and US 2010/0087732, the content of each of which is incorporated by reference herein in its entirety. Additional optical-acoustic sensors are shown for example in U.S. Pat. No. 6,659,957; U.S. Pat. No. 7,527,594; and US 2008/0119739, the content of each of which is incorporated by reference herein in its entirety.

An exemplary optical-acoustic imaging apparatus includes a photoacoustic transducer and a blazed Fiber Bragg grating. Optical energy of a specific wavelength travels down a fiber core of optical fiber and is reflected out of the optical fiber by the blazed grating. The outwardly reflected optical energy impinges on the photoacoustic material. The photoacoustic material then generates a responsive acoustic impulse that radiates away from the photoacoustic material toward nearby biological or other material to be imaged. Acoustic energy of a specific frequency is generated by optically irradiating the photoacoustic material at a pulse rate equal to the desired acoustic frequency.

The optical-acoustic imaging apparatus utilizes at least one and generally more than one optical fiber, for example but not limited to a glass fiber at least partly composed of silicon dioxide. The basic structure of a generic optical fiber is illustrated in FIG. 18, which fiber generally consists of layered glass cylinders. There is a central cylinder called the core 1. Surrounding this is a cylindrical shell of glass, possibly multilayered, called the cladding 2. This cylinder is surrounded by some form of protective jacket 3, usually of plastic (such as acrylate). For protection from the environment and more mechanical strength than jackets alone provide, fibers are commonly incorporated into cables. Typical cables have a polyethylene sheath 4 that encases the fibers within a strength member 5 such as steel or Kevlar strands.

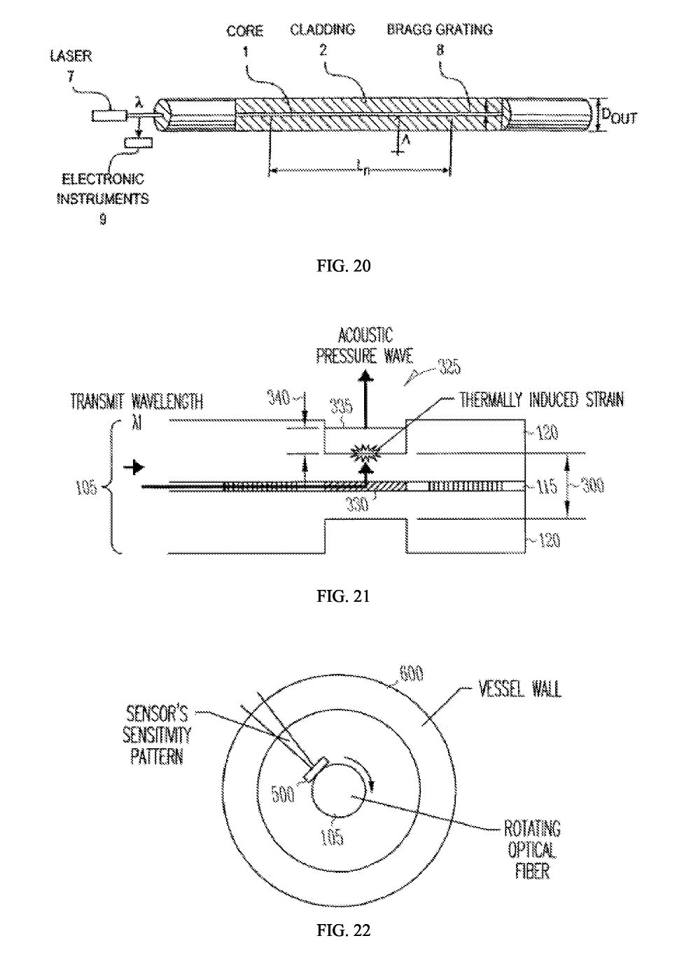

FIG. 19 is a cross-sectional schematic diagram illustrating generally one example of a distal portion of an imaging assembly that combines an acousto-optic Fiber Bragg Grating (FBG) sensor 100 with an photoacoustic transducer 325. The optical fiber includes a blazed Fiber Bragg grating. Fiber Bragg Gratings form an integral part of the optical fiber structure and can be written intracore during manufacture or after manufacture. As illustrated in FIG. 20, when illuminated by a broadband light laser 7, a uniform pitch Fiber Bragg Grating element 8 will reflect back a narrowband component centered about the Bragg wavelength λ given by λ=2nλ, where n is the index of the core of the fiber and λ represents the grating period. Using a tunable laser 7 and different grating periods (each period is approximately 0.5 μm) situated in different positions on the fiber, it is possible to make independent measurement in each of the grating positions.

Referring back to FIG. 19, unlike an unblazed Bragg grating, which typically includes impressed index changes that are substantially perpendicular to the longitudinal axis of the fiber core 115 of the optical fiber 105, the blazed Bragg grating 330 includes obliquely impressed index changes that are at a nonperpendicular angle to the longitudinal axis of the optical fiber 105. As mentioned above, a standard unblazed FBG partially or substantially fully reflects optical energy of a specific wavelength traveling down the axis of the fiber core 115 of optical fiber 105 back up the same axis. Blazed FBG 330 reflects this optical energy away from the longitudinal axis of the optical fiber 105. For a particular combination of blaze angle and optical wavelength, the optical energy will leave blazed FBG 330 substantially normal (i.e., perpendicular) to the longitudinal axis of the optical fiber 105. In the illustrative example of FIG. 22, an optically absorptive photoacoustic material 335 (also referred to as a “photoacoustic” material) is placed on the surface of optical fiber 105. The optically absorptive photoacoustic material 335 is positioned, with respect to the blazed grating 330, so as to receive the optical energy leaving the blazed grating. The received optical energy is converted in the optically absorptive material 335 to heat that expands the optically absorptive photoacoustic material 335. The optically absorptive photoacoustic material 335 is selected to expand and contract quickly enough to create and transmit an ultrasound or other acoustic wave that is used for acoustic imaging of the region of interest.

FIG. 21 is a cross-sectional schematic diagram illustrating generally one example of the operation of photoacoustic transducer 325 using a blazed Bragg grating 330. Optical energy of a specific wavelength, λ₁, travels down the fiber core 115 of optical fiber 105 and is reflected out of the optical fiber 105 by blazed grating 330. The outwardly reflected optical energy impinges on the photoacoustic material 335. The photoacoustic material 335 then generates a responsive acoustic impulse that radiates away from the photoacoustic material 335 toward nearby biological or other material to be imaged. Acoustic energy of a specific frequency is generated by optically irradiating the photoacoustic material 335 at a pulse rate equal to the desired acoustic frequency.

In another example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In yet a further example, the photoacoustic material is of a thickness 300 that is about ¼ the acoustic wavelength of the material at the desired acoustic transmission/reception frequency, and the corresponding glass-based optical fiber sensing region resonant thickness 300 is about ½ the acoustic wavelength of that material at the desired acoustic transmission/reception frequency. This further improves the generation of acoustic energy by the photoacoustic material and reception of the acoustic energy by the optical fiber sensing region.

In one example of operation, light reflected from the blazed grating excites the photoacoustic material in such a way that the optical energy is efficiently converted to substantially the same acoustic frequency for which the FBG sensor is designed. The blazed FBG and photoacoustic material, in conjunction with the aforementioned FBG sensor, provide both a transmit transducer and a receive sensor, which are harmonized to create an efficient unified optical-to-acoustic-to-optical transmit/receive device. In one example, the optical wavelength for sensing is different from that used for transmission. In a further example, the optical transmit/receive frequencies are sufficiently different that the reception is not adversely affected by the transmission, and vice-versa.

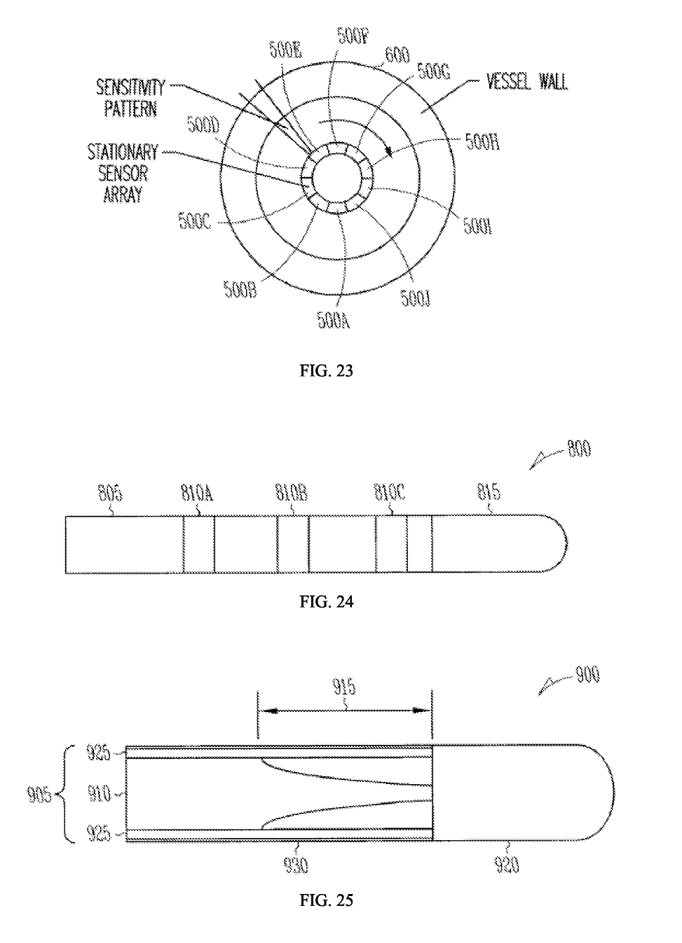

FIG. 22 is a schematic diagram illustrating generally one technique of generating an image of biological material and a vessel wall 600 through an opening in a device. The technique involves rotating the blazed FBG optical-to-acoustic and acoustic-to-optical combined transducer 500 and displaying the resultant series of radial image lines to create a radial image. In another example, phased array images are created using a substantially stationary (i.e., non-rotating) set of multiple FBG sensors, such as FBG sensors 500A-J. FIG. 23 is a schematic diagram that illustrates generally one such phased array example, in which the signal to/from each array transducer 500A-J is combined with the signals from one or more other transducers 500A-J to synthesize a radial image line. In this example, other image lines are similarly synthesized from the array signals, such as by using specific changes in the signal processing used to combine these signals.

FIG. 24 is a schematic diagram that illustrates generally an example of a side view of a distal portion 800 of an elongate device 805. In this example, the distal portion 800 of the device 805 includes one or more openings 810A, 810B, . . . , 810N located slightly or considerably proximal to a distal tip 815 of the device 805. Each opening 810 includes one or more optical-to-acoustic transducers 325 and a corresponding one or more separate or integrated acoustic-to-optical FBG sensors 100. In one example, each opening 810 includes an array of blazed FBG optical-to-acoustic and acoustic-to-optical combined transducers 500 (such as illustrated in FIG. 23) located slightly proximal to distal tip 815 of device 805 having mechanical properties that allow the device 805 to be guided through a vascular or other lumen.

FIG. 25 is a schematic diagram that illustrates generally one example of a cross-sectional side view of a distal portion 900 of another device 905. In this example, optical fibers 925 are distributed around a bottom portion of device 905. In this example, the optical fibers 925 are at least partially embedded in a polymer matrix or other binder material that bonds the optical fibers 925 to the device 905. The binder material may also contribute to the torsion response of the resulting device 905. In one example, the optical fibers 925 and binder material is overcoated with a polymer or other coating 930, such as for providing abrasion resistance, optical fiber protection, and/or friction control.

In one example, before the acoustic transducer(s) is fabricated, the device 905 is assembled, such as by binding the optical fibers 925 to the device 905, and optionally coating the device 905. The opto-acoustic transducer(s) are then integrated into the imaging assembly, such as by grinding one or more grooves in the device wall at locations of the opto-acoustic transducer window 810. In a further example, the depth of these groove(s) in the optical fiber(s) 925 defines the resonant structure(s) of the opto-acoustic transducer(s).

After the opto-acoustic transducer windows 810 have been defined, the FBGs added to one or more portions of the optical fiber 925 within such windows 810. In one example, the FBGs are created using an optical process in which the portion of the optical fiber 925 is exposed to a carefully controlled pattern of UV radiation that defines the Bragg gratings. Then, a photoacoustic material is deposited or otherwise added in the transducer windows 810 over respective Bragg gratings. One example of a suitable photoacoustic material is pigmented polydimethylsiloxane (PDMS), such as a mixture of PDMS, carbon black, and toluene.

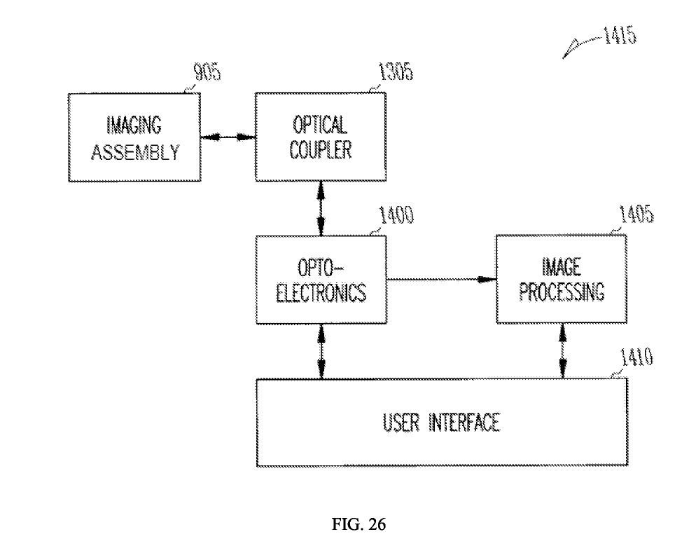

FIG. 26 is a block diagram illustrating generally one example of the imaging assembly 905 and associated interface components. The block diagram of FIG. 26 includes the imaging assembly 905 that is coupled by optical coupler 1305 to an optoelectronics module 1400. The optoelectronics module 1400 is coupled to an image processing module 1405 and a user interface 1410 that includes a display providing a viewable still and/or video image of the imaging region near one or more acoustic-to-optical transducers using the acoustically-modulated optical signal received therefrom. In one example, the system 1415 illustrated in the block diagram of FIG. 26 uses an image processing module 1405 and a user interface 1410 that are substantially similar to existing acoustic imaging systems.

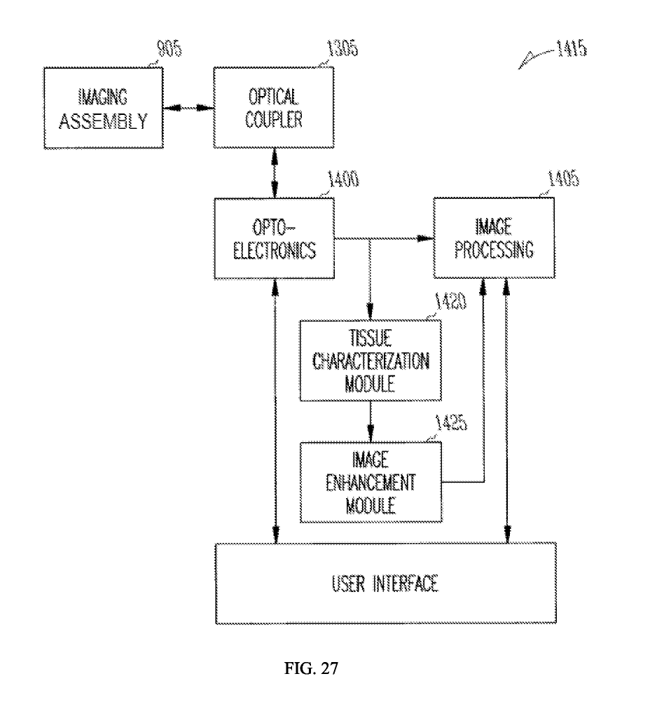

FIG. 27 is a block diagram illustrating generally another example of the imaging assembly 905 and associated interface components. In this example, the associated interface components include a tissue (and plaque) characterization module 1420 and an image enhancement module 1425. In this example, an input of tissue characterization module 1420 is coupled to an output from optoelectronics module 1400. An output of tissue characterization module 1420 is coupled to at least one of user interface 1410 or an input of image enhancement module 1425. An output of image enhancement module 1425 is coupled to user interface 1410, such as through image processing module 1405.

In this example, tissue characterization module 1420 processes a signal output from optoelectronics module 1400. In one example, such signal processing assists in distinguishing plaque from nearby vascular tissue. Such plaque can be conceptualized as including, among other things, cholesterol, thrombus, and loose connective tissue that build up within a blood vessel wall. Calcified plaque typically reflects ultrasound better than the nearby vascular tissue, which results in high amplitude echoes. Soft plaques, on the other hand, produce weaker and more texturally homogeneous echoes. These and other differences distinguishing between plaque deposits and nearby vascular tissue are detected using tissue characterization signal processing techniques.

For example, such tissue characterization signal processing may include performing a spectral analysis that examines the energy of the returned ultrasound signal at various frequencies. A plaque deposit will typically have a different spectral signature than nearby vascular tissue without such plaque, allowing discrimination therebetween. Such signal processing may additionally or alternatively include statistical processing (e.g., averaging, filtering, or the like) of the returned ultrasound signal in the time domain. Other signal processing techniques known in the art of tissue characterization may also be applied. In one example, the spatial distribution of the processed returned ultrasound signal is provided to image enhancement module 1425, which provides resulting image enhancement information to image processing module 1405. In this manner, image enhancement module 1425 provides information to user interface 1410 that results in a displaying plaque deposits in a visually different manner (e.g., by assigning plaque deposits a discernable color on the image) than other portions of the image. Other image enhancement techniques known in the art of imaging may also be applied. In a further example, similar techniques are used for discriminating between vulnerable plaque and other plaque, and enhancing the displayed image provides a visual indicator assisting the user in discriminating between vulnerable and other plaque.

The opto-electronics module 1400 may include one or more lasers and fiber optic elements. In one example, such as where different transmit and receive wavelengths are used, a first laser is used for providing light to the imaging assembly 905 for the transmitted ultrasound, and a separate second laser is used for providing light to the imaging assembly 905 for being modulated by the received ultrasound. In this example, a fiber optic multiplexer couples each channel (associated with a particular one of the optical fibers 925) to the transmit and receive lasers and associated optics. This reduces system complexity and costs.

In one example, the sharing of transmit and receive components by multiple guidewire channels is possible at least in part because the acoustic image is acquired over a relatively short distance (e.g., millimeters). The speed of ultrasound in a human or animal body is slow enough to allow for a large number of transmit/receive cycles to be performed during the time period of one image frame. For example, at an image depth (range) of about 2 cm, it will take ultrasonic energy approximately 26 microseconds to travel from the sensor to the range limit, and back. In one such example, therefore, an about 30 microseconds transmit/receive (T/R) cycle is used. In the approximately 30 milliseconds allotted to a single image frame, up to 1,000 T/R cycles can be carried out. In one example, such a large number of T/R cycles per frame allows the system to operate as a phased array even though each sensor is accessed in sequence. Such sequential access of the photoacoustic sensors in the guidewire permits (but does not require) the use of one set of T/R opto-electronics in conjunction with a sequentially operated optical multiplexer. In one example, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

In one example, in which the imaging assembly 905 includes 30 sequentially-accessed optical fibers having up to 10 photoacoustic transducer windows per optical fiber, 30×10=300 T/R cycles are used to collect the image information from all the openings for one image frame. This is well within the allotted 1,000 such cycles for a range of 2 cm, as discussed above. Thus, such an embodiment allows substantially simultaneous images to be obtained from all 10 openings at of each optical fiber at video rates (e.g., at about 30 frames per second for each transducer window). This allows real-time volumetric data acquisition, which offers a distinct advantage over other imaging techniques. Among other things, such real-time volumetric data acquisition allows real-time 3-D vascular imaging, including visualization of the topology of a blood vessel wall, the extent and precise location of plaque deposits, and, therefore, the ability to identify vulnerable plaque.

In another embodiment, the imaging assembly uses optical coherence tomography (OCT). OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In some embodiments, the imaging assembly is an IVUS imaging assembly. The imaging assembly can be a phased-array IVUS imaging assembly, a pull-back type IVUS imaging assembly and/or rotational IVUS imaging assemblies. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J. C. P. E. 4(2):193 (June 1994), Eberle et al., U.S. Pat. No. 5,453,575, Eberle et al., U.S. Pat. No. 5,368,037, Eberle et al., U.S. Pat. No. 5,183,048, Eberle et al., U.S. Pat. No. 5,167,233, Eberle et al., U.S. Pat. No. 4,917,097, Eberle et al., U.S. Pat. No. 5,135,486, and other references well known in the art relating to intraluminal ultrasound devices and modalities. All of these references are incorporated by reference herein in their entirety.

IVUS imaging is widely used in interventional cardiology as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer. Suitable rotational IVUS catheters include REVOLUTION 45 MHz Catheter (offered by the Volcano Corporation).

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector.

The transducer subassembly can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLO (IVUS fluid flow display software offered by the Volcano Corporation).

Suitable phased array imaging catheters include Volcano Corporation's EAGLE EYE Platinum Catheter, EAGLE EYE Platinum Short-Tip Catheter, and EAGLE EYE Gold Catheter.

The guidewire of the present invention may also include advanced guidewire designs to include sensors that measure flow and pressure, among other things. For example, the FLOWIRE Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PrimeWire PRESTIGE pressure guidewire, available from Volcano Corp. (San Diego, Calif.), provides a microfabricated microelectromechanical (MEMS) pressure sensor for measuring pressure environments near the distal tip of the guidewire. Additional details of guidewires having MEMS sensors can be found in U.S. Patent Publication No. 2009/0088650, incorporated herein by reference in its entirety.

In certain embodiments, angiogram image data is obtained simultaneously with the imaging data obtained from an imaging assembly of the present invention. In such embodiments, the imaging assembly may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

Some exemplary methods of the present invention will now be described. One method of the present invention includes delivering a device to a target site in the body lumen. Once at or near the target site, a slidable cover on the opening is retracted and the imaging assembly is activated. This allows the images of the tissue seen through the opening to be obtained and transmitted back to an operator prior to tissue removal.

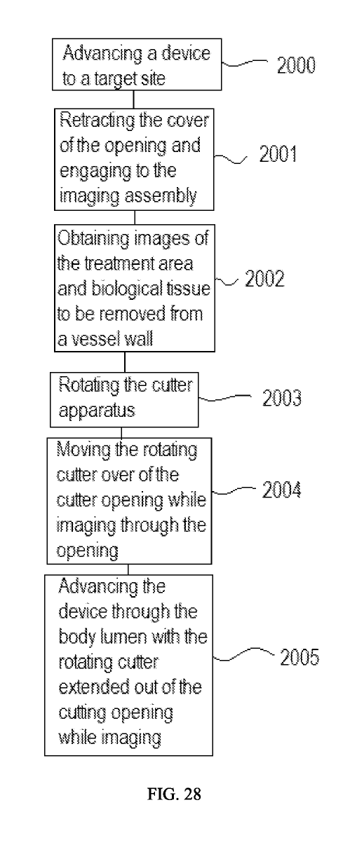

A distal portion of the catheter can be deflected relative to a proximal portion of the catheter to expose a tissue removal assembly in the catheter. Biological material may be removed from the body lumen with the exposed removal assembly while imaging is occurring. Specifically, as shown schematically in FIG. 28, one specific method according to the invention involves advancing a device to a target site (Step 2000). A cover on the opening is retracted and the imaging assembly is used for imaging of the treatment area (e.g., a vessel wall) and biological material to be removed from the treatment area (Step 2001, 2002). In this example, the removal assembly includes a rotatable cutter apparatus. The cutter can be rotated and moved out of the cutting opening while imaging through the opening (Steps 2003, 2004). Preferably, a distal portion of the device can be pivoted or deflected so as to position the cutter adjacent the target material. Thereafter, the device and the rotating cutter can be moved through the body lumen to remove the target material from the body lumen while still imaging the body lumen (Step 2005).

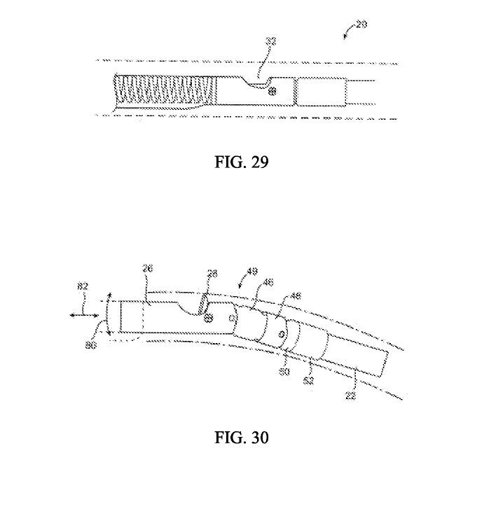

As shown in FIGS. 29 and 30, the device can be percutaneously advanced through a guide catheter or sheath and over a conventional or imaging guidewire using conventional interventional techniques. The device 20 can be advanced over the guidewire and out of the guide catheter to the diseased area. As shown in FIG. 29, the opening 32 will typically be closed (with the cutter or other removal device 28 in a first, distal position). As shown in FIG. 30, device 20 will typically have at least one hinge or pivot connection to allow pivoting about one or more axes of rotation to enhance the delivery of the catheter into the tortuous anatomy without dislodging the guide catheter or other sheath. The cutter can be positioned proximal of the lesion.

Once positioned, the cutter 28 will be retracted proximally and moved out of cutting window 32 to its second, exposed position. In some embodiments, movement of the cutter can deflect the distal portion of the catheter to increase the profile of the catheter at the target site. Movement of the cutter is typically caused by proximal movement of lever 38 and tensioning of drive shaft 36. Movement of the lever can be scaled to any desired ratio or a direct 1:1 ratio of movement between the handle and cutter. When the cutter is moved proximally it contacts ramp or cam surfaces so as to guide the cutter up and at least partially out of the cutting window 32. Additionally, as shown by arrow 80, the distal portion of catheter body 26 rotates about the joint 49 to provide an urging force for the cutter (and catheter body) to move toward the diseased area.

Thereafter, as shown by arrow 82 the operator can move the entire catheter body 22 through the lesion to dissect the tissue. As the cutter 28 and catheter body 22 are advanced distally through the lesion, tissue that is trapped between the cutting edge 52 and the cutting opening 32 is severed from the body lumen. To part off the tissue, the operator can stop pushing the device distally and the cutter can be advanced distally inside the cutting window by advancing the handle 38. During the distal movement of the cutter, the cutter 28 rides back over the ramps 44 and directs the cutter back inside of the cutting window 32. Such movement causes the distal portion 26 of the catheter to move in line with the cutter and proximal portion 24 (FIG. 6B). When the cutter has moved to its distal position, the cutter parts off the severed tissue and urges the severed tissue inside of a collection chamber 53 in the distal tip 42. Optionally, after the cutter 28 has parted off the tissue, the lever 38 and thus the non-rotating cutter 38 can be advanced distally to pack the tissue into the collection chamber 53 (FIG. 6B). Use of the cutter to pack the severed tissue will allow the operator multiple specimens to be collected prior to removing the catheter 20 from the body lumen. When it is determined that the collection chamber is full, the catheter can be removed from the body lumen and the collection chamber can be emptied, and the excised tissue may be stored or tested as described above.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A device for imaging and removing biological material from a vessel wall, the device comprising:
a body configured to fit within a lumen of a vessel, the body comprising an opening;
a biological material removal assembly configured to remove biological material that is exposed to the removal assembly via the opening; and
an imaging assembly coupled to the body and positioned to image the opening.

2. The device according to claim 1, wherein the device is a catheter.

3. The device according to claim 2, wherein the opening is on a side of the catheter.

4. The device according to claim 3, wherein the removal assembly comprises a suction apparatus.

5. The device according to claim 3, wherein the removal assembly comprises a cutter apparatus.

6. The device according to claim 5, wherein the cutter apparatus comprises a rotatable auger.

7. The device according to claim 3, wherein the imaging assembly comprises at least one opto-acoustic sensor.

8. The device according to claim 7, wherein the at least one sensor is placed on an internal wall of the catheter, opposite the opening.

9. The device according to claim 8, wherein the at least one sensor is a plurality of sensors and the sensors are arranged in a semi-circle.

10. The device according to claim 7, wherein the at least one sensor is embedded within an internal wall of the catheter, opposite the opening.

11. The device according to claim 10, wherein the at least one sensor is a plurality of sensors and the sensors are arranged in a semi-circle.

12. The device according to claim 7, wherein the opto-acoustic sensor comprises:
an optical fiber comprising a blazed fiber Bragg grating;
a light source that transmits light through the optical fiber; and
a photoacoustic transducer material positioned so that it receives light diffracted by the blazed fiber Bragg grating and emits ultrasonic imaging energy.

13. The device according to claim 12, wherein the cutter apparatus comprises a rotatable auger.

14. The device according to claim 1, wherein at least a portion of the removal assembly extends through the opening.

15. The device according to claim 1, wherein the opening is configured such that tissue enters the opening and the removal assembly removes the tissue that enters the opening.

16. The device according to claim 1, wherein the biological material is plaque.

17. A method for imaging and removing biological material from a vessel wall, the method comprising:
providing a biological material removal device comprising: a body configured to fit within a lumen of a vessel, the body comprising an opening; a biological material removal assembly configured to remove biological material that is exposed to the removal assembly via the opening; and an imaging assembly coupled to the body and positioned to image the opening;
inserting the device into a lumen of a vessel; and
simultaneously imaging the opening while removing biological material from a vessel wall that is exposed to the removal assembly via the opening.

18. The method according to claim 17, wherein the device is a catheter.

19. The method according to claim 17, wherein the opening is on a side of the catheter.

20. The method according to claim 19, wherein the removal assembly comprises a suction apparatus.

21. The method according to claim 19, wherein the removal assembly comprises a cutter apparatus.

22. The method according to claim 21, wherein the cutter apparatus comprises a rotatable auger.

23. The method according to claim 19, wherein the imaging assembly comprises an opto-acoustic sensor.

24. The method according to claim 23, wherein the at least one sensor is placed on an internal wall of the catheter, opposite the opening.

25. The method according to claim 24, wherein the at least one sensor is a plurality of sensors and the sensors are arranged into a semi-circle.

26. The method according to claim 23, wherein the at least one sensor is embedded within an internal wall of the catheter, opposite the opening.

27. The method according to claim 26, wherein the at least one sensor is a plurality of sensors and the sensors are arranged in a semi-circle.

28. The method according to claim 23, wherein the opto-acoustic sensor comprises:
an optical fiber comprising a blazed fiber Bragg grating;
a light source that transmits light through the optical fiber; and
a photoacoustic transducer material positioned so that it receives light diffracted by the blazed fiber Bragg grating and emits ultrasonic imaging energy.

28. The method according to claim 28, wherein the cutter apparatus comprises a rotatable auger.

30. The method according to claim 17, wherein at least a portion of the removal assembly extends through the opening.

31. The method according to claim 17, wherein the opening is configured such that tissue enters the opening and the removal assembly removes the tissue that enters the opening.

32. The method according to claim 17, wherein the biological material is plaque.
